(19) United States
(12) Patent Application Publication
(10) Pub. No.: US 2018/0368667 A1
(43) Pub. Date: Dec. 27, 2018

(54) VIDEO ENDOSCOPE

(71) Applicant: OLYMPUS WINTER & IBE GMBH, Hamburg (DE)

(72) Inventor: Martin WIETERS, Barsbuettel (DE)

(73) Assignee: OLYMPUS WINTER & IBE GMBH, Hamburg (DE)

(21) Appl. No.: 16/047,138

(22) Filed: Jul. 27, 2018

Related U.S. Application Data
(63) Continuation of application No. PCT/EP2017/052579, filed on Feb. 7, 2017.

(30) Foreign Application Priority Data
Feb. 11, 2016 (DE) 102016202093.5

(51) Int. Cl. A61B 1/04; A61B 1/00; A61B 1/12

(52) U.S. Cl. CPC A61B 1/04; A61B 1/00167; A61B 1/128; A61B 2560/0406; A61B 2562/0271; A61B 2560/0462; A61B 1/00064

(57) ABSTRACT

A video endoscope including: an outer casing tube; an inner casing tube disposed within the outer casing tube; a fiber tube disposed between the outer and inner casing tubes, an image-forming unit being accommodated in the inner casing tube; and a heating film provided between the fiber tube and the inner casing tube.

### CROSS-REFERENCE TO RELATED APPLICATION

The present application is a continuation of PCT/EP2017/052579 filed on Feb. 7, 2017, which is based upon and claims the benefit to DE 10 2016 2020 93.5 filed on Feb. 11, 2016, the entire contents of each of which are incorporated herein by reference.

### BACKGROUND

### Field

The present disclosure relates to a video endoscope and more particularly to a video endoscope having an outer casing tube, a fiber tube for an inner casing tube, and an inner casing tube for an image-forming unit.

### Prior Art

During the assembly of endoscopes, inner tubes that are relatively narrowly designed with regard to their fit are often inserted into an outer casing tube in order to prevent misalignment. In particular with video endoscopes, for example, the video unit, which is also termed an R-unit, is inserted into an outer casing tube. The video unit hereby comprises an inner tube or respectively an inner casing tube into which the optical elements and also a video camera or at least one light-sensitive chip, such as, for example, a CCD chip, are integrated. In order to enable an exact alignment or an exact fixing of the inner tube to the optical axis, the fit to the outer casing tube, or respectively the tube into which the inner tube or respectively the inner casing tube for the image-forming unit is inserted, is relatively narrow.

In particular with video endoscopes, the image-forming unit or respectively the so-called R-unit is inserted into the outer casing tube or respectively into a fiber tube and is proximally fixed. A heat-shrinkable sleeve is pulled over the casing tube for the R-unit, wherein the heat-shrinkable sleeve serves to prevent the inner casing tube from rattling and in addition to also cause an electrical insulation. A heating film is applied to the fiber tube for the inner casing tube circumferentially over the circumference of the fiber tube.

FIG. 1 schematically shows a cross-section through an endoscope shaft 10 of a schematically indicated video endoscope E in detail, wherein the section in FIG. 1 shows a distal end region of the endoscope shaft 10.

The endoscope shaft 10 has an outer casing tube 1 that in the interior encloses a fiber tube 4 encased by a heating film 3. The fiber tube 4 is hereby eccentrically disposed in the outer casing tube 1. Between the fiber tube 4 or respectively the outer heating film 3 and the outer casing tube 1, the intermediate space is filled with optical fibers 2 that end distally in the front face of the video endoscope E.

The fiber tube 4 surrounds an inner casing tube 6 disposed in the interior of the fiber tube 4 for an image-forming unit (not represented here) for the video endoscope E. The image-forming unit can hereby be designed as a so-called R-unit, for example with a CCD chip.

Between the fiber tube 4 and the inner casing tube 6, a heat-shrinkable sleeve 5 is disposed that surrounds the inner casing tube 6.

To prevent a distal window (not represented here) of the video endoscope E from fogging, the fiber tube 4 is enclosed by the heating film 3, which is wrapped around the outer face of the fiber tube 4.

The heating film 3 is hereby attached via a cable (not represented here) in order to subject a heating resistor in the heating film 3 to electrical current, whereby the heating film 3 and the fiber tube 4 enclosed by the heating film 3 are heated so that a front-side window of the video endoscope E is heated.

### SUMMARY

It is the object to enable cost-effective assembly of video endoscopes, wherein it is possible to provide more installation space for other constituent parts of the endoscope if necessary.

such object can be solved by a video endoscope with an outer casing tube, wherein a fiber tube for an inner casing tube is accommodated in the outer casing tube, wherein an inner casing tube for an image-forming unit is accommodated in the fiber tube and wherein a heating film, which can surround the inner casing tube, is provided between the fiber tube and the inner casing tube.

The video endoscope has an outer casing tube and an inner casing tube for an image-forming unit. Furthermore, a fiber tube for the inner casing tube is provided, wherein the fiber tube is accommodated in the outer casing tube and the inner casing tube is accommodated in the fiber tube. In addition, a heating film that can surround the inner casing tube is disposed between the fiber tube in which the inner casing tube is accommodated and the inner casing tube.

In the video endoscope, a heating film instead of the previous heat-shrinkable sleeve is arranged on the inner casing tube for the image-forming unit or respectively the R-unit so that the heating film is or will be arranged between the inner casing tube and the fiber tube surrounding the inner casing tube, so that the heating film is used not only for heating but in addition is also designed as protection against rattling for the inner casing tube. Furthermore, the heating film can also be used as electrical insulation. This depends on the geometry of the installation space in the endoscope shaft or respectively in the outer casing tube.

By disposing the heating film (instead of the previous heat-shrinkable sleeve) between the fiber tube and the inner casing tube, the heat-shrinkable sleeve is omitted during assembly of the video endoscope, whereby a component (heat-shrinkable sleeve) is dispensed with. In addition, this simplifies the assembly of the endoscope since an assembly step is dispensed with. Moreover, with the omission of the heat-shrinkable sleeve, a reduction of the diameter is achieved so that more installation space is created, for example for optical fibers or the optical system, or more stable tubes can be used. In addition, it is possible, due to the reduction of the diameter, to provide video endoscopes that have a smaller diameter of the endoscope shaft.

The heating film heats, for example, a distal window of an endoscope in order to avoid fogging of the window. By means of the heating film, an electrical resistance heating is provided that can be disposed in the vicinity of the window in the endoscope shaft. A good heat transfer through the fiber tube to the window, which is soldered, for example, in the fiber tube that can be provided in good heat contact, is achieved by the contact of the heating film with the fiber tube, which can be formed of or can be produced from metal.

The inner casing tube can have a tube section, wherein the tube section of the inner casing tube is enclosed by the heating film in the circumferential direction. The heating film thereby surrounds the tube section of the inner casing tube.

In addition, the e heating film can be configured in a sleeve-shape or as a heating film sleeve.

The heating film can be in immediate or direct contact with the fiber tube and with the inner casing tube.

The heating film can be produced from plastic, such as polyimide. Heating films made of Kapton® can also be used.

In addition, in one embodiment of the video endoscope it is further provided that an optical fiber bundle can be accommodated in the outer casing tube next to the fiber tube for the inner casing tube.

At least one temperature sensor for detecting the temperature of the heating film can be provided, wherein the heating film can have the at least one temperature sensor. By means of the temperature sensor, the temperature of the heating film in the endoscope shaft is detected or established so that it is possible to set a predetermined temperature by means of a control device or a control circuit depending on the detected heating film temperature.

A temperature control device for controlling the temperature of the heating film can be provided. The temperature control device can be connected to a temperature sensor of the heating film or for the heating film in order to receive a measured actual temperature from the temperature sensor so that a predetermined temperature of the heating film is set or adjusted by means of an actual/set value comparison in the temperature control device connected to the heating film.

The heating film can have a positive temperature coefficient of electrical resistance.

### BRIEF DESCRIPTION OF THE DRAWINGS

Further features will become apparent from the description of the embodiments together with the claims and the attached drawings. Embodiments can fulfill individual features or a combination of several features.

The embodiments are described below, without restricting the general idea, using exemplary embodiments with reference to the drawings, wherein we expressly refer to the drawings with regard to all details that are not explained in greater detail in the text. In the figures:

FIG. 1 schematically illustrates a cross-section through a distal end region of an endoscope shaft according to the prior art, and

FIG. 2 schematically illustrates a cross-section through a distal end region of an endoscope shaft.

FIG. 3 schematically illustrates a cross-section through a distal end region of an endoscope shaft with a temperature control.

In the drawings, in each case the same or similar elements and/or parts are provided with the same reference numbers, so that in each case a repeated introduction is omitted.

### DETAILED DESCRIPTION

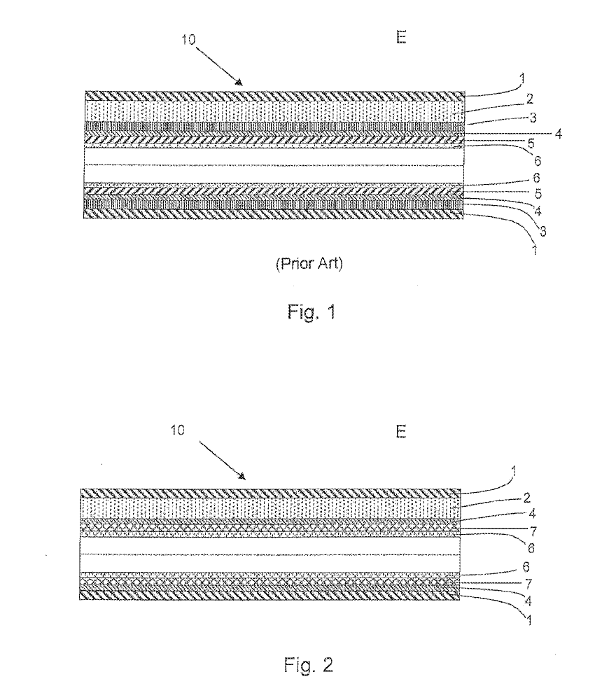

In FIG. 2, a section of an endoscope shaft 10 of a video endoscope E is schematically represented in cross-section. The distal end region of the endoscope shaft 10 is shown schematically in FIG. 2. A fiber tube 4 is disposed in the interior of the outer casing tube 1, wherein the elongated inner casing tube 6 is disposed in the interior of the fiber tube 4. Between the fiber tube 4 and the inner casing tube 6, a heating film 7, which may be a foil, is disposed that is in direct contact with the fiber tube 4 and the inner casing tube 6. The heating film 7 can be configured from Kapton®. In addition, corresponding attaching means for the resistance heating of the heating film 7 are also provided in order to cause a warming of the fiber tube 4.

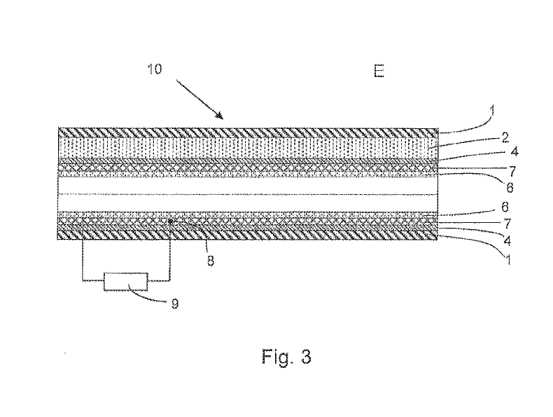

In FIG. 3, a cross-section through an endoscope shaft is schematically represented, wherein, in contrast to the exemplary embodiment in FIG. 2, a temperature control for the heating film 7 is possible. The heating film 7 has a temperature sensor 8, by means of which the temperature of the heating film 7 is detected. When electric current is applied to the heating film 7, it is warmed so that the temperature of the heating film 7 established by the temperature sensor 8 is transmitted to a temperature control device 9, such as a controller, computer or CPU. After an actual/set value comparison in the temperature control device 9, the strength of the electric current for the heating film 7 is correspondingly controlled so that a preferred temperature in the heating film is set and held constant.

While there has been shown and described what is considered to be preferred embodiments, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### REFERENCE SIGN LIST

1 Outer casing tube
2 Optical fibers
3 Heating film
4 Fiber tube
5 Heat-shrinkable sleeve
6 Inner casing tube
7 Heating film
8 Temperature sensor
9 Temperature control device
10 Endoscope shaft
E Video endoscope

What is claimed is:

1. A video endoscope comprising:
n outer casing tube;
an inner casing tube disposed within the outer casing tube;
a fiber tube disposed between the outer and inner casing tubes, an image-forming unit being accommodated in the inner casing tube; and
a heating film provided between the fiber tube and the inner casing tube.

2. The video endoscope according to claim 1, wherein the heating film surrounds the inner casing tube.

3. The video endoscope according to claim 1, wherein the inner casing tube has a tube section, wherein the tube section of the inner casing tube is enclosed by the heating film in a circumferential direction.

4. The video endoscope according to claim 1, wherein the heating film is configured in a sleeve-shape.

5. The video endoscope according to claim 1, wherein the heating film is in direct contact with the fiber tube and with the inner casing tube.

6. The video endoscope according to claim 1, wherein the heating film is formed from plastic.

7. The video endoscope according to claim 6, wherein the plastic is polyimide.

8. The video endoscope according to claim 1, further comprising an optical fiber bundle provided between the outer casing tube and the fiber tube.

9. The video endoscope according to claim 1, further comprising at least one temperature sensor operatively arranged in the video endoscope for detecting the temperature of the heating film.

10. The video endoscope according to claim 9, wherein the at least one temperature sensor is arranged in the heating film.

11. The video endoscope according to claim 9, further comprising a controller for controlling the temperature of the heating film based on an output of the at least one temperature sensor.

12. The video endoscope according to claim 1, wherein the heating film has a positive temperature coefficient of electrical resistance.
